(19) United States
(12) Patent Application Publication
(10) Pub. No.: US 2007/0129676 A1
(43) Pub. Date: Jun. 7, 2007

(54) Safe syringe

(76) Inventor: Hsiu-Chih Lin, Taichung Hsien (TW)

Correspondence Address: COOPER & DUNHAM, LLP, 1185 AVENUE OF THE AMERICAS, NEW YORK, NY, 10036, US

(21) Appl. No.: 11/294,274

(22) Filed: Dec. 5, 2005

(51) Int. Cl. A61M 5/00

(52) U.S. Cl. 604/110; 604/197

(57) ABSTRACT

A safe syringe has a barrel, a needle assembly and a plunger. The barrel has an open front end, an inner surface end and an inner thread formed on the inner surface at the open front end. The needle assembly is mounted retractably in the open front end of the barrel and has a tapered hub and a needle. The tapered hub has a front, an open back, an axial recess and at least one longitudinal groove. The needle is mounted on the tapered hub. The plunger is mounted slidably through the open back end of the barrel. The needle assembly is rotated and pulled into the barrel by a friction force between the plunger and tapered hub of the needle assembly, so medical personnel can safely deal with the used safe syringe.

### BACKGROUND OF THE INVENTION

1. Field of the Invention

The present invention relates to a syringe, and more particularly to a safe syringe that prevents medical personnel from being injured by a syringe needle when medical personnel handle and dispose of used syringes.

2. Description of Related Art

Syringes are generally used to inject liquid medication into a human body. Conventional syringes usually have a barrel, a plunger and a needle cap. The barrel has a front end, an open back end, a medication chamber and a needle. The medication chamber is defined in the barrel and communicates with the open back end. The needle is hollow, is mounted through the front end of the barrel and has a longitudinal passage that communicates with the medication chamber. The plunger is mounted slidably through the open back end and into the medication chamber and has a front end, a back end, a rod and a sealed piston. The sealed piston is mounted on the front end of the plunger and slidably inside the medication chamber. The needle cap is mounted detachably on the front end of the barrel and covers the needle. After the syringe is used, medical personnel such as doctors or nurses put the needle cap back on the barrel to cover the used needle and to keep any person from injuring by the used needle. However, the medical personnel may be stabbed inadvertently by the contaminated needle when the needle cap is reinstalled on the front of the barrel and contract a disease.

Many types of safety syringes are designed for preventing the above problems. However, the conventional safety syringes have complex structures, so to make the conventional safety syringes needs precisely molds and that increases the cost of the safety syringes and is time consuming. The above factors cause the conventional syringes not be used in widely.

To overcome the shortcomings, the present invention provides a safe syringe to mitigate or obviate the aforementioned problems.

### SUMMARY OF THE INVENTION

The main objective of the invention is to provide a safe syringe that prevents medical personnel from being hurt by contaminated needles when medical personnel handle contaminated syringes.

A safe syringe in accordance with the present invention comprises a barrel, a needle assembly and a plunger.

The barrel has an open front end, an open back end and multiple longitudinal slots defined through the barrel and communicate with the open front end.

The needle assembly is mounted retractably in the open front end of the barrel and has a tapered hub and a needle. The tapered hub has a front, an open back, an axial recess and at least one longitudinal groove. The needle is mounted on the tapered hub.

The plunger is mounted slidably through the open back end of the barrel.

The needle assembly is pulled into the barrel, the plunger is rotated to release the tapered hub from the barrel by a friction force between the plunger and tapered hub of the needle assembly, so medical personnel can safely deal with the used safe syringe.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a safe syringe in accordance with the present invention;

FIG. 2 is a cross sectional side view of the safe syringe in FIG. 1;

FIG. 3 is a cross sectional side view of the safe syringe in FIG. 1 with a stopper on a plunger being partially mounted inside the needle assembly;

FIG. 4 is a cross sectional side view of the safe syringe in FIG. 1 with a stopper on a plunger being completely mounted inside the needle assembly;

FIG. 5 is an operational cross sectional side view of the safe syringe in FIG. 2 with the plunger being rotated to release needle assembly from the barrel; and

FIG. 6 is an operational cross sectional side view of the safe syringe in FIG. 2 with the plunger being broken at the annular snapping groove.

### DETAILED DESCRIPTION OF PREFERRED EMBODIMENT

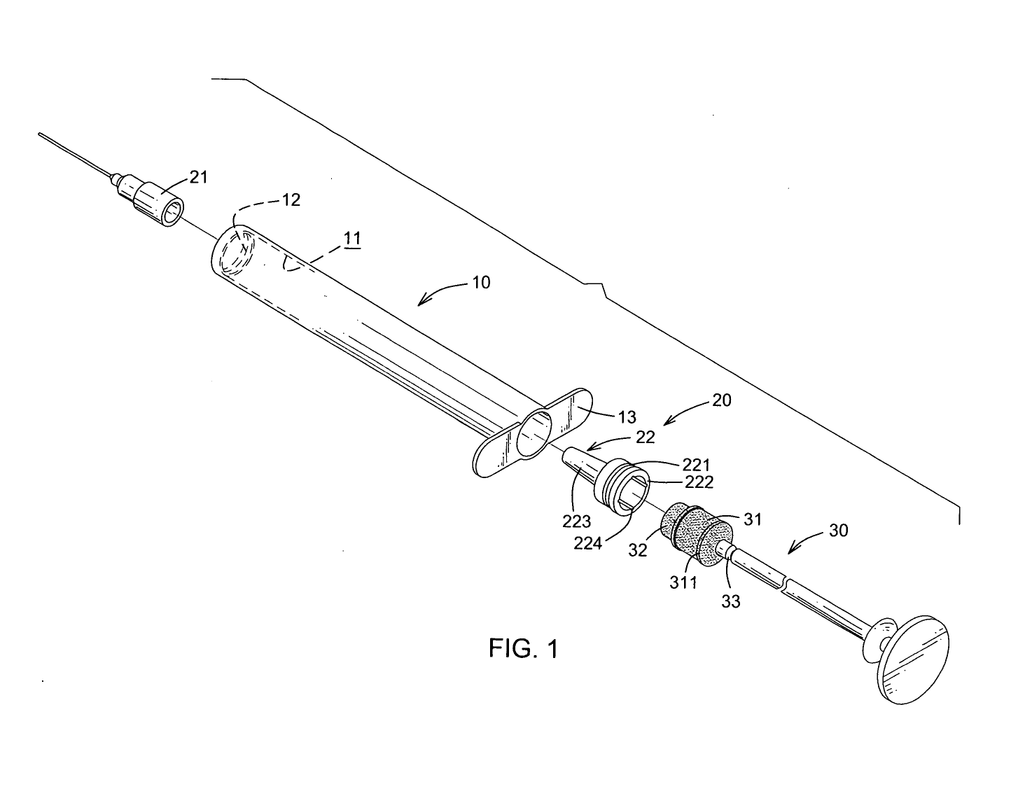

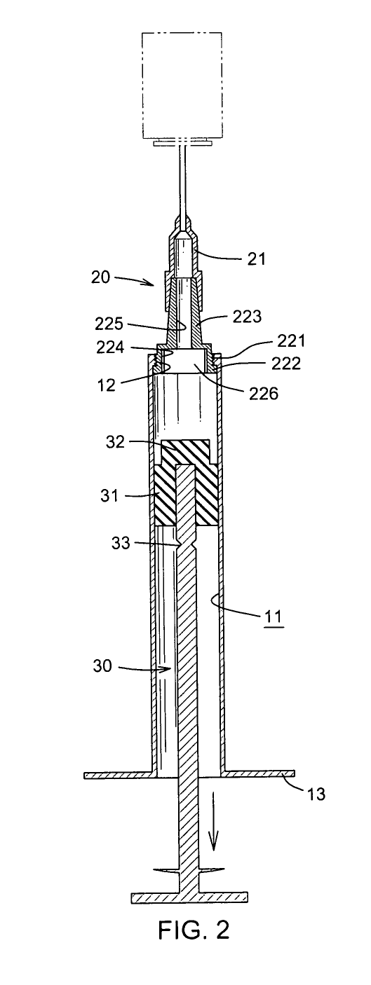

With reference to FIGS. 1 and 2, a safe syringe in accordance with the present invention comprises a barrel (10), a needle assembly (20) and a plunger (30).

The barrel (10) is made of resilient material such as plastic and has an open front end, an open back end, a medication chamber (11), an outer surface, an inner surface, an inner thread (12) and two opposite finger tabs (13).

The medication chamber (11) is defined inside the barrel (10) and communicates with the open front end and the open back end. The inner thread (12) is formed on the inner surface at the open front end of the barrel (10). The finger tabs (13) extend out radially from the open back end of the barrel (10).

The needle assembly (20) is mounted retractably through the open front end of the barrel (10) and has a needle (21) and a tapered hub (22).

The tapered hub (22) is mounted slidably in the open front end of the barrel (10) and has an open back, an outer surface, an inner surface, an outer thread (221), an annular flange seal (222), a tapered front (223), at least one longitudinal groove (224), a central fluid channel (225) and an axial recess (226).

The open back of the tapered hub (22) communicates with the medication chamber (11) in the barrel (10) and extends out the open front end of the barrel (10). The outer thread (221) is formed on the outer surface of the tapered hub (22) and corresponds to the inner thread (12) in the inner surface of the barrel (10). The annular flange seal (222) extends out radially from the open back of the tapered hub (22), abuts the inner surface of the barrel (10) and forms a seal between the barrel (10) and the tapered hub (22). The axial recess (226) is defined inside the tapered hub (22), communicates with the central fluid channel (225) and the open back and has a depth. The multiple longitudinal grooves (224) are defined longitudinally in the inner surface of the tapered hub (22) and communicate with the axial recess (226) and the open back. Each longitudinal groove (224) has a length equal to the depth of the axial recess (226). The central fluid channel (225) is defined through the tapered front (223) of the tapered hub (22). A preferred embodiment of the tapered hub (22) has four longitudinal grooves (224).

The needle (21) is mounted on the tapered front (223) of the tapered hub (22) and has a needle passage. The needle passage is defined axially through the needle and communicates with the central fluid channel (225) in the tapered front (223).

The plunger (30) is mounted slidably through the open back end of the barrel (10) and has a front end, a back end, an outer surface, a plug (31), a sealed piston (32) and an optional annular snapping groove (33). The plug (31) is cylindrical, mounted slidably in the medication chamber (11) and has a front end. The sealed piston (32) is securely formed on the front end of the plug (31) and corresponds to the axial recess (226) in the tapered hub (22). The sealed piston (32) is made of resilient material such as rubber and is mounted on and slides with the plunger (30). The annular snapping groove (33) is defined in the outer surface near the front end of the plunger (30).

With reference to FIG. 2, to draw liquid medication into the medication chamber (11), the plunger (40) is pulled back so the liquid medication passes through the needle passage in the needle (21), the central fluid channel (225), the axial recess (226) and the longitudinal grooves (224) in the tapered hub (22) and finally flows into the medication chamber (11) in the barrel (10). Then, the needle (21) is oriented upward and the plunger (30) is pushed slightly into the barrel (10) to discharge the remaining air out from the barrel (10).

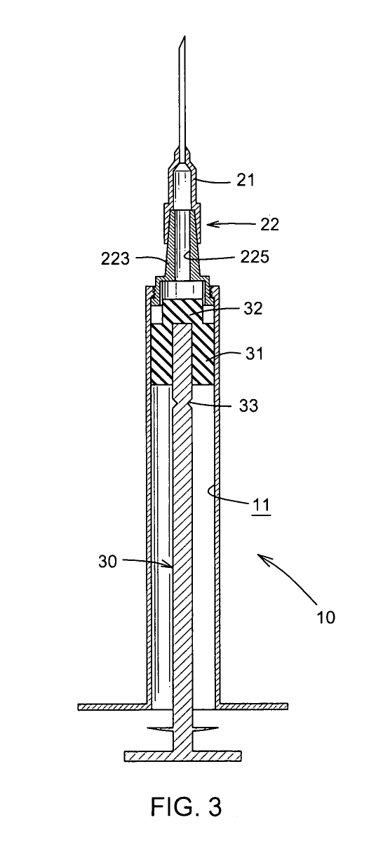

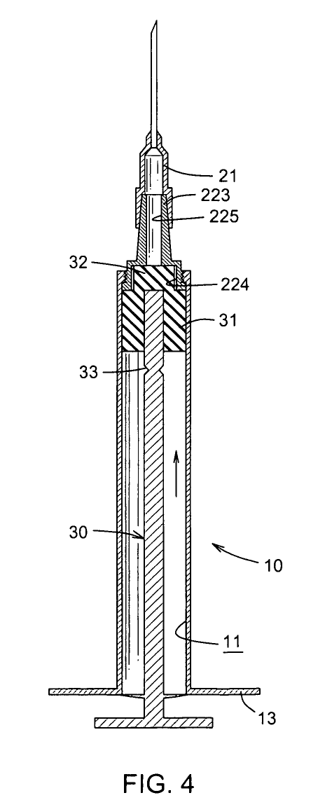

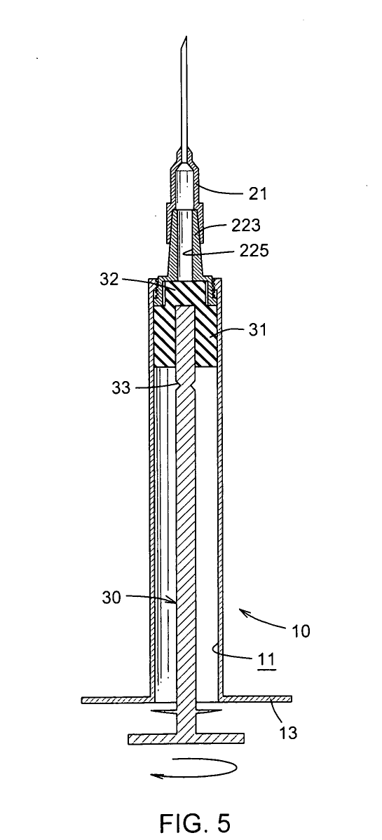

With reference to FIG. 3, when the safe syringe inject medication into a patient, the plunger (30) is pressed to push the liquid medication out of the needle via the axial recess (226), the central fluid channel (225) and the needle passage in the needle (21). When the sealed piston (32) is partially extended into axial recess (226) in tapered hub (22) and seals the axial recess (226), the medication flows from the medication chamber (11) to the central fluid channel (225) through the longitudinal grooves (224). With reference to FIGS. 4 and 5, when the sealed piston (32) on the plug (31) is completely extended into the axial recess (226), the seal piston (32) will tightly engage with the axial recess (226) of the tapered hub (22) with friction. At this time, the sealed piston (32) abuts and seals the open back end of the tapered hub (22) and closes the longitudinal grooves (224). Then, the plunger (30) is rotated to make the tapered hub (22) to rotate with the plunger (30) and to release from the barrel (10) by friction between the sealed piston (32) and the axial recess (226). Consequently, the needle (21) can be pulled into the medication chamber (11).

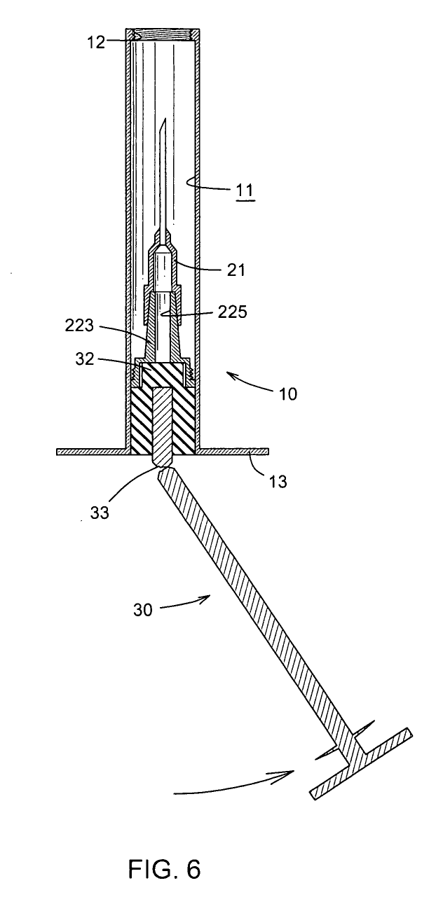

With further reference to FIG. 6, when the annular snapping groove (33) reaches the open rear end of the barrel (10), the plunger (30) is snapped off at the annular snapping groove (33) to prevent the needle assembly (20) from extending out of the open front end of the barrel (10).

With the needle assembly (20) retracted into the barrel (10), medical personnel can safely deal with the used safe syringe without fear of injury from the contaminated needle.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A safe syringe comprising:
a barrel having
an open front end;
an open back end;
a medication chamber defined inside the barrel and communicating with the open front end and the open back end;
an inner surface;
an inner thread formed on the inner surface at the open front end of the barrel; and
two opposite finger tabs extending out radially from the open back end of the barrel;
a needle assembly mounted retractably through the open front end of the barrel and having
a tapered hub mounted slidably in the open front end of the barrel and having
an open back communicating with the medication chamber in the barrel;
an outer surface;
an inner surface;
a length;
an inner thread;
a tapered front;
at least one longitudinal groove defined longitudinally in the inner surface of the tapered hub and communicating with the axial recess and the open back;
a central fluid channel defined through the tapered front of the tapered hub; and
an axial recess defined inside the tapered hub communicating with the central fluid channel, the at least one longitudinal groove and the open back; and
a needle mounted on the tapered front of the tapered hub and having a needle passage defined axially through the needle and communicating with the central fluid channel in the tapered hub;
a plunger mounted slidably through the open back end of the barrel and having
a front end;
a back end;
a plug being cylindrical, mounted slidably in the medication chamber of the barrel and having a front end; and
a sealed piston made of resilient material, mounted securely around the front end of the plug and mounted slidably in the axial recess in the tapered hub.

2. The safe syringe as claimed in claim 1, wherein the tapered hub further has an annular flange seal extending out radially from the open back of the tapered hub and forming a seal between the barrel and the tapered hub.

3. The safe syringe as claimed in claim 1, wherein the plunger further has an annular snapping groove defined in the outer surface near the front end

4. The safe syringe as claimed in claim 2, wherein the plunger further has an annular snapping groove defined in the outer surface near the front end of the plunger.

5. The safe syringe as claimed in claim 4, wherein the tapered hub has four longitudinal grooves.

6. The safe syringe as claimed in claim 1, wherein the tapered hub has four longitudinal grooves.
